(19) United States
(12) Patent Application Publication
(10) Pub. No.: US 2020/0279645 A1
(43) Pub. Date: Sep. 3, 2020

(54) MEDICAL CARE SUPPORT DEVICE, MEDICAL CARE SUPPORT METHOD, AND MEDICAL CARE SUPPORT PROGRAM

(71) Applicant: FUJIFILM CORPORATION, Tokyo (JP)

(72) Inventors: Shigetoshi ISHIKAWA, Kanagawa (JP); Yasuhisa KANEKO, Kanagawa (JP); Haruyasu NAKATSUGAWA, Kanagawa (JP)

(21) Appl. No.: 16/785,654

(22) Filed: Feb. 10, 2020

(30) Foreign Application Priority Data
Feb. 28, 2019 (JP) 2019-036589

(51) Int. Cl. G16H 40/67; G16H 70/60; G16H 50/20

(52) U.S. Cl. CPC G16H 40/67; G16H 50/20; G16H 70/60

(57) ABSTRACT

Provided are a medical care support device, a medical care support method, and a medical care support program capable of improving efficiency of medical care. A medical care support device includes an output unit that outputs a format that is support information for supporting acquisition, by an owner of a pet, of medical condition information indicating a medical condition of a disease from which the pet suffers, and an acquiring unit that acquires medical condition information acquired by the owner on the basis of the format.

### CROSS-REFERENCE TO RELATED APPLICATIONS

This application is based on and claims priority under 35 USC 119 from Japanese Patent Application No. 2019-036589 filed on Feb. 28, 2019, the disclosure of which is incorporated herein by reference in its entirety.

### BACKGROUND OF THE INVENTION

### 1. Field of the Invention

The present disclosure relates to a medical care support device, a medical care support method, and a computer-readable storage medium storing a medical care support program.

### 2. Description of the Related Art

Generally, before a medical examination is performed by a doctor, a patient writes and submits health information on an interview sheet, or the like.

JP2003-022325A discloses a technique for receiving an input form in which health information related to a user is input from an information terminal device of the user and transmitting information related to a disease corresponding to the received health information.

The input format disclosed in JP2003-022325A is a format that conforms to a so-called questionnaire, and is used to input information such as a past history, habits, physical information, or symptoms. The information filled in the questionnaire is general information about health, and it is not always possible to obtain information necessary for medical care according to a disease. In general, the questionnaire is submitted before the first examination, for example, there are cases where the questionnaire is insufficient to obtain information for medical care after the first examination.

In particular, in a case where a patient that suffers from a disease is a pet, since it is difficult for the pet itself to send information necessary for medical care to an owner or a veterinarian, sufficient information may not be transmitted to the doctor.

As a result, pet care by veterinarians tends to be inefficient.

### SUMMARY OF THE INVENTION

The present disclosure has been made in consideration of the above-mentioned problems, and an object of the present disclosure is to provide a medical care support device, a medical care support method, a terminal device, and a computer-readable storage medium storing a medical care support program capable of improving efficiency of medical care.

In order to achieve the above object, according to a first aspect of the present disclosure, there is provided a medical care support device that includes a processor that is configured to: output support information for supporting acquisition, by an owner of a pet, of medical condition information indicating a medical condition of a disease from which the pet suffers; and acquire information of the pet acquired by the owner on the basis of the support information.

Further, in order to achieve the above object, according to an eighth aspect of the present disclosure, there is provided a medical care support method including: by one or more processors: outputting support information for supporting acquisition, by an owner of a pet, of medical condition information indicating a medical condition of a disease from which the pet suffers; and acquiring information of the pet acquired by the owner on the basis of the support information.

In addition, in order to achieve the above object, according to a ninth aspect of the present disclosure, there is provided a computer-readable storage medium storing a medical care support program for causing a computer to: output support information for supporting acquisition, by an owner of a pet, of medical condition information indicating a medical condition of a disease from which the pet suffers; and acquire information of the pet acquired by the owner on the basis of the support information.

Furthermore, according to a tenth aspect of the present disclosure, a terminal device incudes: a display device that displays support information for supporting acquisition, by a user, of medical condition information indicating a medical condition of a disease from which the pet suffers; and an input device that accepts input information from the user on the basis of the support information.

### BRIEF DESCRIPTION OF THE DRAWINGS

FIG. 1 is a block diagram showing an example of a configuration of a medical care support system according to an embodiment.

FIG. 2 is a block diagram showing an example of a hardware configuration of the medical care support device according to the embodiment.

FIG. 3 is a block diagram showing an example of a configuration of an information storage unit according to the embodiment.

FIG. 4 is a diagram showing an example of identification information/disease name correspondence information according to the embodiment.

FIG. 5 is a diagram showing an example of disease name/medical condition information correspondence information according to the embodiment.

FIG. 6 is a diagram showing an example of format information according to the embodiment.

FIG. 7 is a diagram showing an example of accumulated medical condition information according to the embodiment.

FIG. 8 is a block diagram showing an example of a hardware configuration of an owner terminal according to the embodiment.

FIG. 9 is a block diagram showing an example of a hardware configuration of a veterinarian terminal according to the embodiment.

FIG. 10 is a functional block diagram showing an example of a configuration of a medical care support device according to the embodiment.

FIG. 11 is a flowchart showing an example of a medical care supporting process executed by the medical care support device according to the embodiment.

FIG. 12 is a flowchart showing an example of a medical condition information acquiring process executed by the owner terminal according to the embodiment.

FIG. 13 is a diagram showing an example of a state in which a format is displayed on a display unit of the owner terminal according to the embodiment.

FIG. 14 is a diagram showing an example of a state in which a format is displayed on the display unit of the owner terminal according to the embodiment.

FIG. 15 is a diagram showing an example of a state in which a format is displayed on the display unit of the owner terminal according to the embodiment.

FIG. 16 is a diagram showing an example of a state in which a format is displayed on the display unit of the owner terminal according to the embodiment.

FIG. 17 is a diagram showing an example of a state in which a format is displayed on the display unit of the owner terminal according to the embodiment.

FIG. 18 is a flowchart showing an example of a progress observing process executed by the veterinarian terminal according to the embodiment.

FIG. 19 is a flowchart showing an example of a medical examination requesting process executed by the veterinarian terminal according to the embodiment.

### DESCRIPTION OF THE PREFERRED EMBODIMENTS

Hereinafter, exemplary embodiments for performing the technique of the present disclosure will be described in detail with reference to the accompanying drawings.

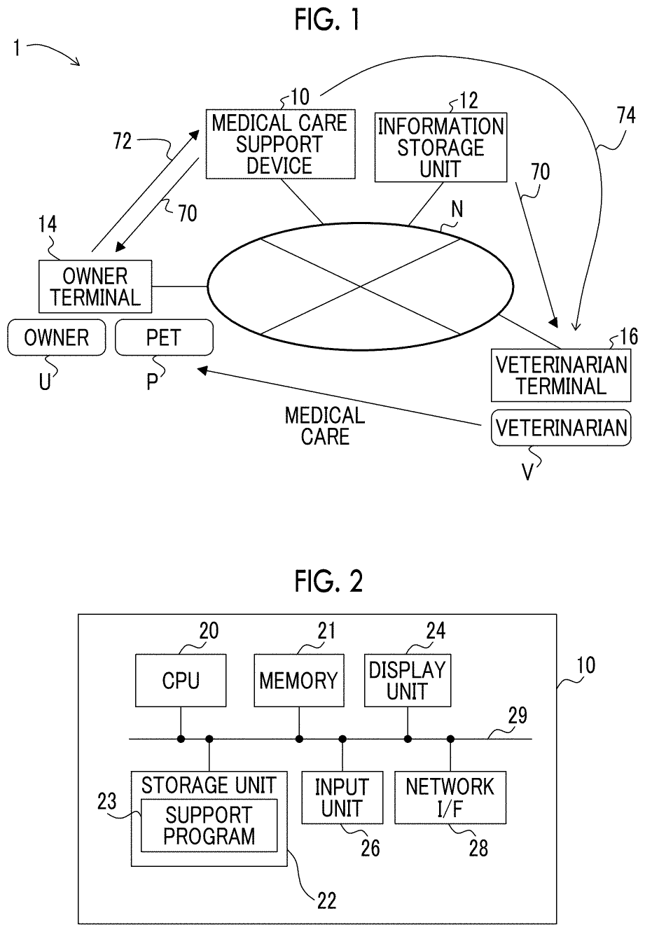

First, a medical care support system 1 of an embodiment will be described with reference to FIG. 1. The medical care support system 1 of this embodiment supports pet medical care performed by a veterinarian in an animal hospital or the like. In this embodiment, the “medical care” is used as a general term for “medical examination”, “diagnosis”, “treatment”, and the like. Hereinafter, as a specific example, a case where medical care in the next medical examination is supported, for a pet P examined by a veterinarian V at an animal hospital, by supporting progress observation by an owner U of the pet P until the next medical examination (re-examination), will be described.

FIG. 1 shows a block diagram showing an example of a configuration of the medical care support system 1 of the present embodiment. As shown in FIG. 1, the medical care support system 1 of this embodiment includes a medical care support device 10, an information storage unit 12, an owner terminal 14, and a veterinarian terminal 16. The medical care support device 10, the information storage unit 12, the owner terminal 14, and the veterinarian terminal 16 are connected to a network N, respectively, and is able to communicate with each other through a network N. The present disclosure is not limited to this embodiment, and a configuration in which at least one of the medical care support device 10, the information storage unit 12, the owner terminal 14, or the veterinarian terminal 16 communicates with other devices without going through the network N may be used.

The medical care support device 10 has a function for providing the owner U with support information (hereinafter, referred to as a “format”) for supporting the owner U of the pet P that is a medical care target in acquiring information related to a medical condition of the pet P (hereinafter, referred to as “medical condition information”) as a progress observation. Specifically, the medical care support device 10 outputs a format for acquiring medical condition information to the owner terminal 14 owned by the owner U. Further, the medical care support device 10 has a function for acquiring medical condition information acquired on the basis of a format provided from the medical care support device 10 from the owner terminal 14. The acquired medical condition information is accumulated in the information storage unit 12.

An example of a hardware configuration of the medical care support device 10 of this embodiment will be described with reference to FIG. 2. As shown in FIG. 2, the medical care support device 10 includes a central processing unit (CPU) 20, a memory 21 that serves as a temporary storage area, and a non-volatile storage unit 22. Further, the medical care support device 10 includes a display unit 24 such as a liquid crystal display, an input unit 26 such as a keyboard or a mouse, and a network interface (I/F) 28 connected to the network N. The CPU 20, the memory 21, the storage unit 22, the display unit 24, the input unit 26, the network I/F 28 are connected to a bus 29 so that they can communicate with each other.

The storage unit 22 is realized by a hard disk drive (HDD), a solid state drive (SSD), a flash memory, and the like. A support program 23 is stored in the storage unit 22 that serves as a storage medium. The CPU 20 reads out the support program 23 from the storage unit 22, develops the support program 23 into the memory 21, and executes the developed support program 23.

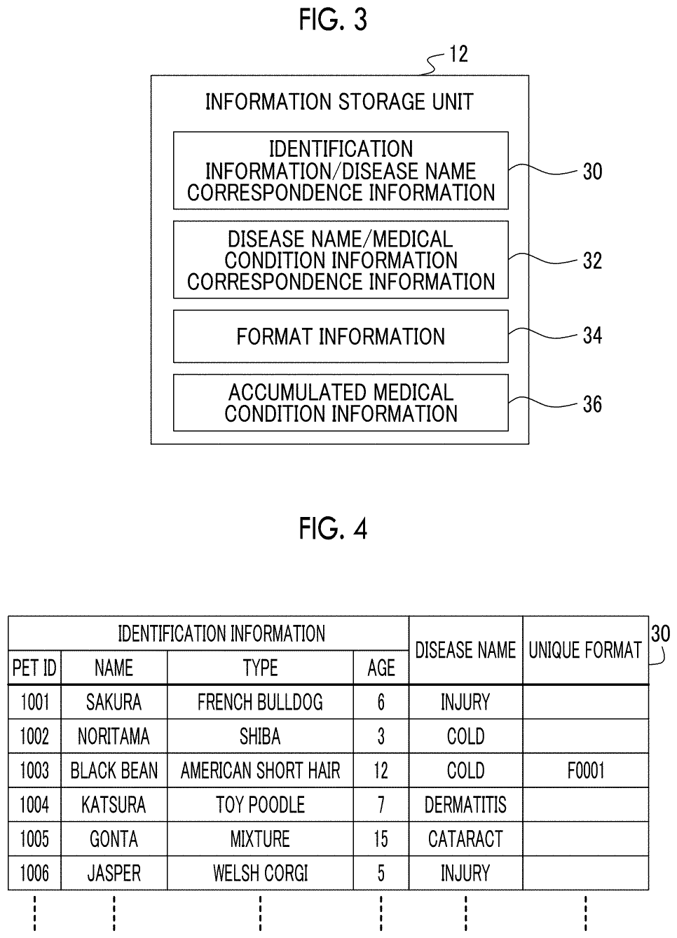

Further, the information storage unit 12 of this embodiment is a cloud-type external storage that is able to be used online. As shown in FIG. 3, identification information/disease name correspondence information 30, disease name/medical condition information name correspondence information 32, format information 34, and accumulated medical condition information 36 are stored in the information storage unit 12.

First, the identification information/disease name correspondence information 30 will be described. FIG. 4 shows an example of the identification information/disease name correspondence information 30. The identification information/disease name correspondence information 30 is information representing a correspondence relationship between identification information for identifying the pet P and a disease name of the pet P.

In the example of the identification information/disease name correspondence information 30 shown in FIG. 4, as the identification information of the pet P, a “pet ID (Identifier)”, a “name” of the pet P, a “type” of the pet P, and an “age” of the pet P are exemplified. The “type” of the pet P includes species or breed of the pet P. In addition, the present disclosure is not limited to this embodiment, and for example, information relating to the owner U may also be used as the identification information. Further, the “disease name” in the example of the identification information/disease name correspondence information 30 is not limited to a “disease name”, but may also include “injury”, or the like, and additionally, may include a “disease name” or a “symptom name”.

The identification information and the disease name may be provided in such a form that, for example, the medical care support device 10 acquires identification information input through the veterinarian terminal 16 by the veterinarian V and registers the result in the identification information/disease name correspondence information 30. Further, for example, in a case where the veterinarian V's animal hospital uses an electronic medical record, a form in which the medical care support device 10 acquires identification information from the electronic medical record of the pet P and registers the result in the identification information/disease name correspondence information 30 may be used.

Further, as shown in FIG. 4, information indicating the presence or absence of unique format information is registered in the identification information/disease name correspondence information 30 of the present embodiment. Although details will be described later, as an example, in the medical care support system 1 of this embodiment, a default format is predetermined for each disease (disease name), but the format may be customized according to a desire of the veterinarian V. Hereinafter, the format customized for each pet P by the veterinarian V may be referred to as a “unique format”. In the example of FIG. 4, a state where a unique format information ID “F0001” is registered for a pet P of which a pet ID is “1003” is shown.

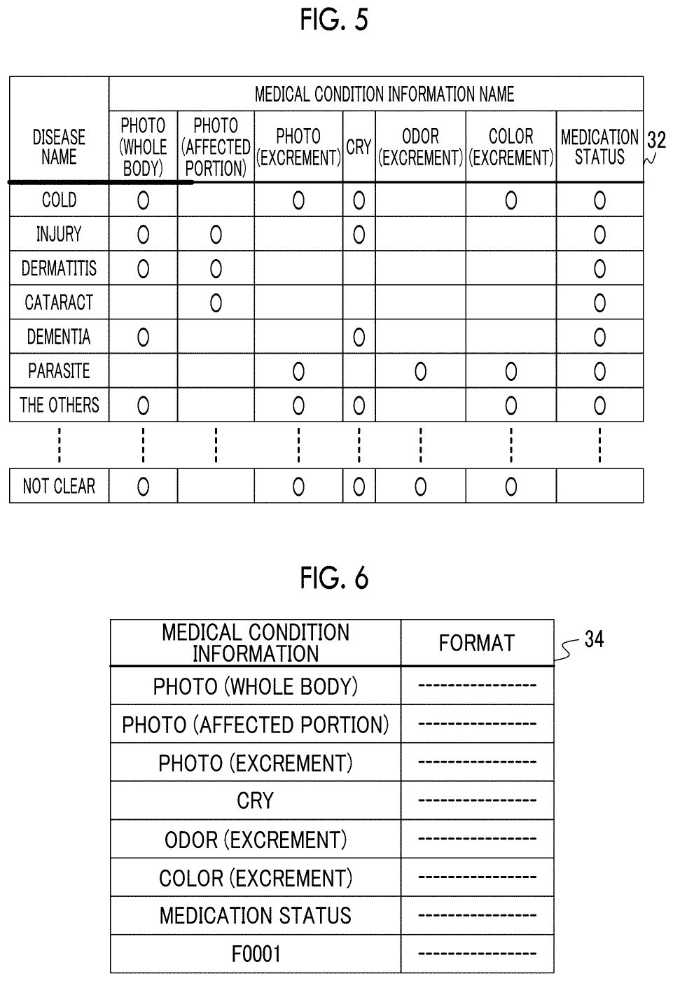

Next, the disease name/medical condition information name correspondence information 32 will be described. FIG. 5 shows an example of the disease name/medical condition information name correspondence information 32. The disease name/medical condition information name correspondence information 32 is information representing the correspondence relationship between a disease name and a medical condition information name necessary for medical care and progress observation of the disease. The disease name/medical condition information name correspondence information 32 of this embodiment is an example of a correspondence of the present disclosure.

The medical condition information is information related to the medical condition of the pet P as a progress observation as described above, which is information for medical care of a disease from which the pet P suffers. The medical condition information includes at least one of information related to an appearance of the pet, information related to a behavior of the pet, and information related to excrement. In general, a symptom referred to by a veterinarian in medical examination, a portion where the symptom appears, content of a progress observation, and the like may differ depending on diseases. That is, the medical condition information necessary for medical care may differ for each disease.

For example, in a case where the pet P suffers from a cold, symptoms may appear in the whole body by repeating a state of crying, a state of excrement, fatigue and sneezing, or the like. Further, for example, in a case where the pet P is injured, it is necessary to observe a state of the affected portion or a state of the whole body, and in a case where the pet is in pain, a symptom may appear in a cry, for example.

In addition, in the case of colds, sore throats, tracheal collapse, injuries, urolithiasis, dementia, and viral rhinotracheitis (in the case of cats), symptoms often appear in a cry. Further, in the case of colonic diarrhea, anal cystitis, hemorrhage, parvovirus infection, or the like, symptoms may appear in excrement (stool) through symptoms such as bloody stool, which may show red. Similarly, as diseases that show symptoms in the color of excrement (stool), gastric ulcer and small intestinal diarrhea, tumors, ulcers, parasites, and the like tend to show black excrement, and infection by protozoa such as Giardia and Trichomonas tends to show yellow excrement. Further, parasites, diarrhea due to infection, intestinal bacterial flora, abnormal digestive functions, and the like tend to show green excrement, and gallbladder disorders, Giardia, acute hepatitis, pancreatitis, cirrhosis, and the like tend to show white or gray excrement. Further, as diseases in which symptoms appear in the odor of excrement (stool), there are chronic enteritis, pancreatitis, and the like. Furthermore, as diseases in which symptoms appear in the frequency of excrement (urine), there is hyperthyroidism, or the like.

Further, depending on the above-mentioned diseases, symptoms may appear in the form of excrement (stool) such as solid and mud, and symptoms may appear in the amount of excrement. In addition, in a case where the pet P vomits due to the above-mentioned diseases, the vomit (included in the excrement) may include information necessary for medical care of the diseases.

In this way, there is a characteristic in a portion where a symptom appears due to a disease. Further, in addition to a portion where a symptom appears, there is also medical condition information necessary or useful for medical care. For example, in the case of dermatitis, since the spread of inflammation, in addition to an affected portion itself, is information necessary for medical care, the state of the whole body of the pet P is also information that is useful for medical care. On the other hand, there is a case where the state of the affected portion itself is information necessary for medical care, such as cataract.

Further, as an element that affects progression of a disease, in other words, change of a symptom, there is a medication status of the pet P, for example.

Thus, in the medical care support system 1 of the present embodiment, for each disease, information in which a disease name and a medical condition information name that is necessary or useful for medical care and a progress observation of the disease are associated with each other is used as the disease name/medical condition information name correspondence information 32.

FIG. 5 shows an example in which a “photo (whole body)” that represents an image of the whole body of the pet P, a “photo (affected portion)” that represents an image of an affected portion, a “photo (excrement)” that represents an image of excrement of the pet P, a “cry” that represents a sound of a cry of the pet, a “odor (excrement)” that represents a odor of the excrement of the pet P, a “color (excrement)” that represents a color of the excrement of the pet P, and “medication information” are used as medical condition information.

In the example shown in FIG. 5, in a case where the disease name is “injury”, “photo (whole body)”, “photo (affected portion)”, “cry”, and “medication status” are associated as medical condition information necessary for a progress observation.

In addition, depending on the type of an animal is, for example, whether the animal is a dog or a cat, or depending on the breed or the like of a dog in a case where the animal is the dog, there is a case where the type of a disease from which the animal suffers or a symptom to appear differs. Accordingly, it is preferable to prepare a plurality of types of disease name/medical condition information name correspondence information 32 in accordance with the type, breed, and the like of the animal.

Next, the format information 34 will be described. FIG. 6 shows an example of the format information 34. In the format information 34, data of each format is stored for each piece of medical condition information. In the format information 34, data of each format itself may be stored, or a location where the format exists, such as a URL (Uniform Resource Locator), may be stored. Further, as shown in FIG. 6, in the format information 34, data of the above-described unique format is also stored.

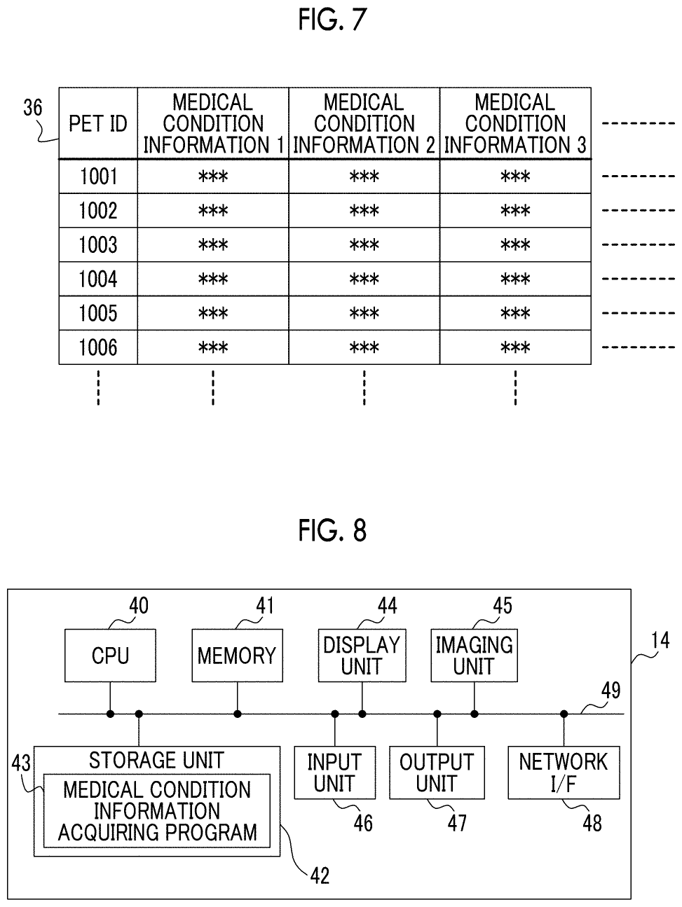

Next, the accumulated medical condition information 36 will be described. FIG. 7 shows an example of the accumulated medical condition information 36. The accumulated medical condition information 36 is accumulated in a state where medical condition information acquired by the medical care support device 10 from the owner terminal 14 is associated with information for identifying the pet P (a pet ID in the example shown in FIG. 7).

On the other hand, as the owner terminal 14, for example, a smartphone, a tablet computer, or the like may be used. In this embodiment, a configuration in which the owner terminal 14 is formed as a single device has been described, but a configuration in which the owner terminal 14 is formed as a plurality of devices may be used.

With reference to FIG. 8, an example of a hardware configuration of the owner terminal 14 of the present embodiment will be described. As shown in FIG. 8, the owner terminal 14 includes a CPU 40, a memory 41 that is a temporary storage area, and a non-volatile storage unit 42. Further, the owner terminal 14 includes a display unit 44 such as a liquid crystal display, an imaging unit 45 such as a camera, an input unit 46 such as a microphone that is a sound collecting device, an output unit 47 such as a speaker, and a network I/F 48 connected to the network N. The CPU 40, the memory 41, the storage unit 42, the display unit 44, the imaging unit 45, the input unit 46, the output unit 47, and the network I/F 48 are connected to a bus 49 so as to be able to communicate with each other. As an example, the owner terminal 14 of this embodiment includes a touch panel display having functions of the display unit 44 and the input unit 46.

The storage unit 42 is realized by an HDD, an SSD, a flash memory, and the like. A medical condition information acquiring program 43 is stored in the storage unit 42 as a storage medium. The CPU 40 reads out the medical condition information acquiring program 43 from the storage unit 42, develops the medical condition information acquiring program 43 in the memory 41, and executes the developed medical condition information acquiring program 43.

On the other hand, the veterinarian terminal 16 includes a desktop computer, a tablet computer, or the like, for example. In this embodiment, a configuration in which the veterinarian terminal 16 is formed as a single device has been described, but a configuration in which the veterinarian terminal 16 is formed as a plurality of devices may be used.

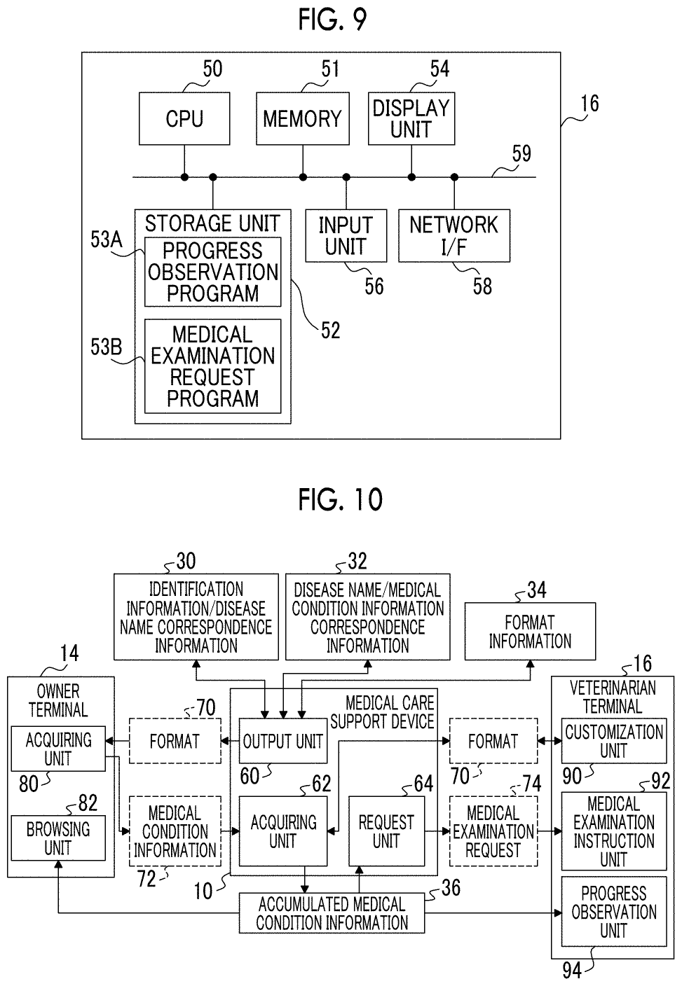

With reference to FIG. 9, an example of a hardware configuration of the veterinarian terminal 16 of the present embodiment will be described. As shown in FIG. 9, the veterinarian terminal 16 includes a CPU 50, a memory 51 that is a temporary storage area, and a non-volatile storage unit 52. Further, the veterinarian terminal 16 includes a display unit 54 such as a liquid crystal display, an input unit 56 such as a keyboard, a mouse, or a microphone, and a network I/F 58 connected to the network N. The CPU 50, the memory 51, the storage unit 52, the display unit 54, the input unit 56, and the network I/F 58 are connected to a bus 59 so as to be able to communicate with each other.

The storage unit 52 is realized by an HDD, an SSD, a flash memory, and the like. Progress observation program 53A and medical examination request program 53B are stored in the storage unit 52 that is a storage medium. The CPU 50 reads out each of the progress observation program 53A and the medical examination request program 53B from the storage unit 52, develops the progress observation program 53A and the medical examination request program 53B in the memory 41, and executes the developed progress observation program 53A and medical examination request program 53B.

Next, with reference to FIG. 10, functional configurations of the medical care support device 10, the owner terminal 14, and the veterinarian terminal 16 according to the present embodiment will be described. FIG. 10 shows a functional block diagram showing an example of configurations of the medical care support device 10, the owner terminal 14, and the veterinarian terminal 16 of this embodiment.

As shown in FIG. 10, the medical care support device 10 includes an output unit 60, an acquiring unit 62, and a request unit 64. The CPU 20 of the medical care support device 10 executes the support program 23, the CPU 20 functions as the output unit 60, the acquiring unit 62, and the request unit 64. Further, the owner terminal 14 includes an acquiring unit 80 and a browsing unit 82. As a CPU 40 of the owner terminal 14 executes the medical condition information acquiring program 43, the CPU 40 functions as the acquiring unit 80 and the browsing unit 82. Further, the veterinarian terminal 16 includes a customization unit 90, a medical examination instruction unit 92, and a progress observation unit 94. As a CPU 50 of the veterinarian terminal 16 executes the progress observation program 53A, the CPU 50 functions as the customization unit 90 and the progress observation unit 94, and as the CPU 50 executes the medical examination request program 53B, the CPU 50 functions as the medical examination instruction unit 92.

The output unit 60 of the medical care support device 10 outputs a format 70 for supporting acquisition of medical condition information 72 necessary for a progress observation of the pet P, with reference to the identification information/disease name correspondence information 30, the disease name/medical condition information name correspondence information 32, and the format information 34. The format 70 output from the output unit 60 is transmitted to the owner terminal 14 through the network N by the network I/F 28.

The format 70 transmitted from the medical care support device 10 is acquired by the acquiring unit 80 of the owner terminal 14. The owner U acquires the medical condition information 72 on the basis of the format 70 acquired by the acquiring unit 80. The acquiring unit 80 of the owner terminal 14 transmits the acquired medical condition information 72 from the owner terminal 14 to the medical care support device 10 through the network N.

The acquiring unit 62 of the medical care support device 10 acquires the medical condition information 72 transmitted from the owner terminal 14 through the network I/F 28. The acquiring unit 62 accumulates the acquired medical condition information 72 in the accumulated medical condition information 36.

Further, the output unit 60 of the medical care support device 10 outputs the format 70 in response to a request from the veterinarian terminal 16. The format 70 output from the output unit 60 is transmitted to the veterinarian terminal 16 through the network N by the network I/F 28.

The format 70 transmitted from the medical care support device 10 is acquired by the customization unit 90 of the veterinarian terminal 16. The format 70 used for the progress observation by the veterinarian V can be customized by the customization unit 90 of the veterinarian terminal 16 of the present embodiment. Accordingly, the customization unit 90 of the veterinarian terminal 16 acquires a predetermined format 70 such as a default format 70 from the medical care support device 10, and then, outputs the customized format 70 to the medical care support device 10 by the veterinarian V.

On the other hand, the request unit 64 of the medical care support device 10 requests the veterinarian V for medical care on the basis of the medical condition information 72 accumulated in the accumulated medical condition information 36. For example, the request unit 64 of this embodiment transmits a medical care request 74 to the veterinarian terminal 16 through the network N by the network I/F 28 at a predetermined timing (which will be described later in detail).

The medical care request 74 transmitted from the medical care support device 10 is received by the medical examination instruction unit 92 of the veterinarian terminal 16. On the basis of the received medical care request 74, the medical examination instruction unit 92 prompts the veterinarian V to examine the pet P based on the medical condition information 72 accumulated in the accumulated medical condition information 36.

Further, the browsing unit 82 of the owner terminal 14 of this embodiment acquires the medical condition information 72 accumulated in the accumulated medical condition information 36, and displays the medical condition information 72 to be browsed by the owner U.

In addition, the progress observation unit 94 of the veterinarian terminal 16 of the present embodiment acquires the medical condition information 72 accumulated in the accumulated medical condition information 36 in a case where the veterinarian V observes the progress information of the pet P that is a medical care target, and displays the medical condition information 72 to be observed by the veterinarian V.

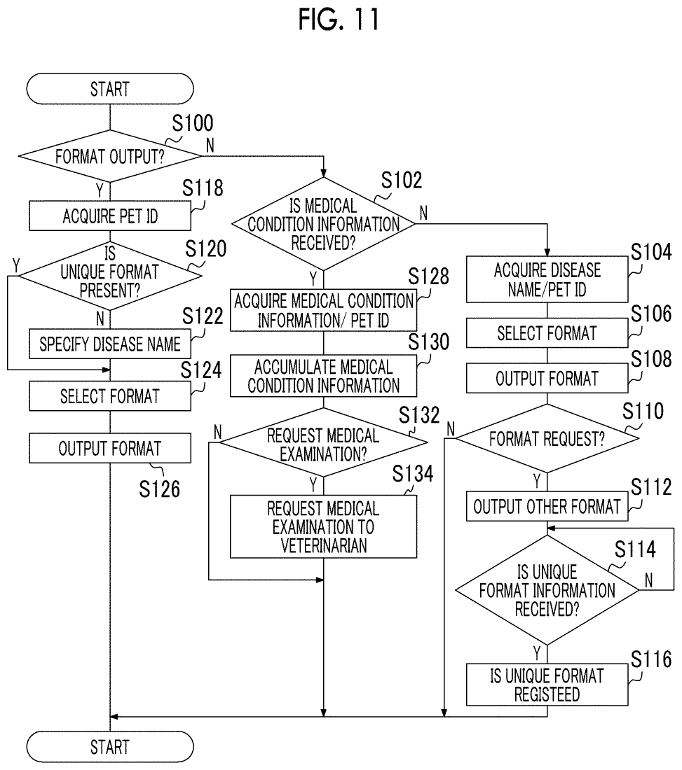

Next, an operation of the medical care support system 1 according to this embodiment will be described. First, an operation of the medical care support device 10 will be described with reference to FIG. 11. In this embodiment, the CPU 20 of the medical care support device 10 executes the support program 23 so that a medical care supporting process shown in FIG. 11 is executed. As an example, in the medical care supporting process of the present embodiment shown in FIG. 11, in a case where the pet ID is received from the owner terminal 14, the format 70 corresponding to the pet P corresponding to the pet ID is transmitted to the owner terminal 14. Further, in the medical care supporting process of this embodiment, in a case where the medical condition information 72 and the pet ID are received from the owner terminal 14, the medical condition information 72 is accumulated in the information storage unit 12. In addition, in the medical care supporting process of this embodiment, in a case where the disease name or pet ID is received from the veterinarian terminal 16, the format 70 is customized by the veterinarian terminal 16. Accordingly, in the medical care support device 10 of this embodiment, the medical care supporting process shown in FIG. 11 is executed in a case where any of the pet ID, the medical condition information 72, and a disease name is received from the owner terminal 14 or the veterinarian terminal 16.

In step S100 of FIG. 11, the output unit 60 determines whether to output the format 70. In the medical care support device 10 of this embodiment, in a case where the pet ID is received from the owner terminal 14 as described above and the medical condition information 72 is not received, the format 70 is output. Accordingly, in a case where the medical condition information 72 is received from the owner terminal 14 and in a case where the pet ID is not received from the owner terminal 14, the determination in step S100 is negative, and the procedure proceeds to step S102.

In step s102, the output unit 60 determines whether or not the medical condition information 72 has been received from the owner terminal 14. In a case where the medical condition information 72 has not been received from the owner terminal 14, in other words, in a case where the disease name or the pet ID has been received from the veterinarian terminal 16, the determination in step S102 is negative, and the procedure proceeds to step S104.

In step S104, the output unit 60 acquires the received disease name or pet ID. In the next step S106, the output unit 60 acquires the format 70 corresponding to the received disease name or pet ID from the information storage unit 12. Specifically, in a case where the disease name is acquired from the veterinarian terminal 16, the output unit 60 specifies a medical condition information name corresponding to the received disease name, with reference to the disease name/medical condition information name correspondence information 32 in the information storage unit 12. Further, the output unit 60 acquires the format 70 corresponding to the specified medical condition information name from the format information 34. On the other hand, in a case where the pet ID is acquired from the veterinarian terminal 16, the output unit 60 specifies unique format information corresponding to the received pet ID, with reference to the identification information/disease name correspondence information 30 in the information storage unit 12. Further, the output unit 60 acquires the format 70 corresponding to the specified unique format information from the format information 34.

In the next step S108, the output unit 60 outputs the format 70 acquired in step S106. The format 70 output from the output unit 60 is transmitted to the veterinarian terminal 16 through the network N.

In the veterinarian terminal 16 that has received the format 70 by the customization unit 90, the format 70 is customized by determination of the veterinarian V. In a case where the veterinarian V customizes the format 70, a request for another format 70 is made from the customization unit 90.

Accordingly, the output unit 60 determines whether or not a request for the format 70 is received from the veterinarian terminal 16 in the next step S110. In a case where the request for the format 70 has not been received, the determination in step S110 is negative, and the present medical care supporting process is terminated. On the other hand, in a case where the request for the format 70 is received from the veterinarian terminal 16, the determination in step S110 is affirmative, and the procedure proceeds to step S112.

In step S112, the output unit 60 outputs another format 70 that is different from the format 70 output in step S108. Specifically, the output unit 60 acquires and outputs the format 70 that has not been acquired in the process of step S108 from the format information 34 of the information storage unit 12. The other format 70 output from the output unit 60 is transmitted to the veterinarian terminal 16 through the network N.

Although details will be described later, the customization unit 90 of the veterinarian terminal 16 that has received the other format 70 uses the other format 70 to customize the format 70 by determination of the veterinarian V. In a case where the veterinarian V customizes the format 70, the customized format 70 is transmitted from the customization unit 90 as information indicating the unique format 70 (hereinafter, referred to as “unique format information”). As an example, in the present embodiment, the unique format information includes information indicating which format 70 is included.

Accordingly, in step S114, the output unit 60 determines whether or not the unique format information (format 70) has been received from the veterinarian terminal 16. The determination in step S114 is negative until the unique format information (format 70) is received from the veterinarian terminal 16. On the other hand, in a case where the unique format information (format 70) is received from the veterinarian terminal 16, the determination in step S114 is affirmative, and the procedure proceeds to step S116.

In step S116, the output unit 60 registers the received unique format information (format 70), and then, ends the present medical care supporting process. Specifically, in a case where the processes of steps S104 to S114 are executed by receiving the disease name from the veterinarian terminal 16, the output unit 60 changes the medical condition information name associated with the disease name, of the disease name/medical condition information name correspondence information 32 in the information storage unit 12, in accordance with the unique format information. Further, in a case where the processes of steps S104 to S114 are executed by receiving the pet ID from the veterinarian terminal 16, the output unit 60 assigns an ID for identifying the unique format information to the received unique format information (format 70), and associates the assigned ID with the pet ID of the identification information/disease name correspondence information 30 in the information storage unit 12 (see FIG. 4 and the “unique format” of the identification information/disease name correspondence information 30). Then, the output unit 60 registers the unique format in the format information 34.

As described above, in the medical care supporting process of this embodiment, the format 70 corresponding to the veterinarian V can be customized by the processes of steps S104 to S116.

On the other hand, in a case where the medical care support device 10 receives the pet ID from the owner terminal 14, the determination in step S100 is affirmative, and the procedure proceeds to step S118. In step S118, the output unit 60 acquires the pet ID received from the owner terminal 14.

In the next step S120, the output unit 60 determines whether or not the unique format information is associated with the acquired pet ID. Specifically, the output unit 60 determines whether or not the unique format information is associated with the acquired pet ID, with reference to the identification information/disease name correspondence information 30 in the information storage unit 12. In a case where the unique format information is associated with the acquired pet ID, the determination in step S120 is affirmative, and the procedure proceeds to step S124. On the other hand, in a case where the unique format information is not associated with the acquired pet ID, the determination in step S120 is negative, and the procedure proceeds to step S122.

In step S122, the output unit 60 specifies a disease name corresponding to the received pet ID. In other words, the disease name in which the pet P having the acquired pet ID is affected is specified. Specifically, the output unit 60 specifies the disease name associated with the acquired pet ID, with reference to the identification information/disease name correspondence information 30 in the information storage unit 12.

In the next step S124, the output unit 60 selects the format 70 to be output. Specifically, in a case where the determination in step S120 is affirmative and the procedure proceeds to step S124, the output unit 60 selects the format 70 corresponding to the unique format information from the format information 34. Further, in a case where the disease name is specified in step S122, the output unit 60 specifies a medical condition information name corresponding to the disease name specified from the disease name/medical condition information name correspondence information 32. In addition, the output unit 60 selects the format 70 corresponding to the specified medical condition information name from the format information 34.

In the next step S126, the output unit 60 outputs the format 70 selected in step S124 to the owner terminal 14, and then, terminates the present medical care supporting process. The format 70 output from the output unit 60 is transmitted to the owner terminal 14 through the network N.

In the acquiring unit 80 of the owner terminal 14 that has received the format 70, the medical condition information 72 is acquired by the owner U on the basis of the format 70.

On the other hand, in a case where the medical care support device 10 has received the medical condition information 72 from the owner terminal 14, the determination in step S102 is affirmative, and the procedure proceeds to step S128. In step S128, the acquiring unit 62 acquires the medical condition information 72 and the pet ID received from the owner terminal 14.

In the next step S130, the acquiring unit 62 accumulates the acquired medical condition information 72 in the accumulated medical condition information 36 of the information storage unit 12 in association with the pet ID.

In the next step S132, the request unit 64 determines whether or not to request the veterinarian V for medical examination. In the medical care support device 10 of the present embodiment, the veterinarian V is requested to examine the medical condition information 72 according to the acquired medical condition information 72. As an example, in a case where the medical condition information 72 is received from the owner terminal 14 a predetermined number of times, the request unit 64 of the present embodiment requests the veterinarian V to examine the pet P based on the medical condition information 72. In this way, by requesting medical examination from the veterinarian V, before the pet P visits an animal hospital, it is possible to perform the medical examination by the veterinarian V with respect to whether there has been a change in a medical condition during a progress observation, for example, a sudden change, or the like. A timing at which the request unit 64 requests the veterinarian V for medical examination is not limited to this embodiment. For example, a configuration in which in a case where the request unit 64 analyzes the medical condition information 72 accumulated in the accumulated medical condition information 36 and it is determined that there has been a sudden change, the veterinarian V is requested to perform medical examination may be used. In this case, for example, the request unit 64 may perform a request for medical examination according to a determination result, using a learning model for determining sudden change or progression of a disease, in which a combination of data representing a symptom corresponding to the medical condition information 72 (for example, a photo of an affected portion) and a disease is constructed as teacher data.

In a case where the veterinarian V is not requested to perform the medical examination, the determination in step S132 is negative, and the medical care supporting process is terminated. On the other hand, in a case where the veterinarian V is requested to perform the medical examination, the determination in step S132 is affirmative, and the procedure proceeds to step S134.

In step S134, the request unit 64 transmits the medical care request 74 to the veterinarian terminal 16 of the veterinarian V, and then, terminates the medical care supporting process. In addition, the form of the medical examination request transmitted by the request unit 64 is not particularly limited. For example, the request unit 64 may transmit a pet ID and a message indicating a request for medical examination in an e-mail format. Further, for example, unlike the present embodiment, a transmission destination of the medical examination request to be transmitted by the request unit 64 may be a device different from the veterinarian terminal 16, for example, a mobile terminal of the veterinarian V that is owned separately from the veterinarian terminal 16, or the like.

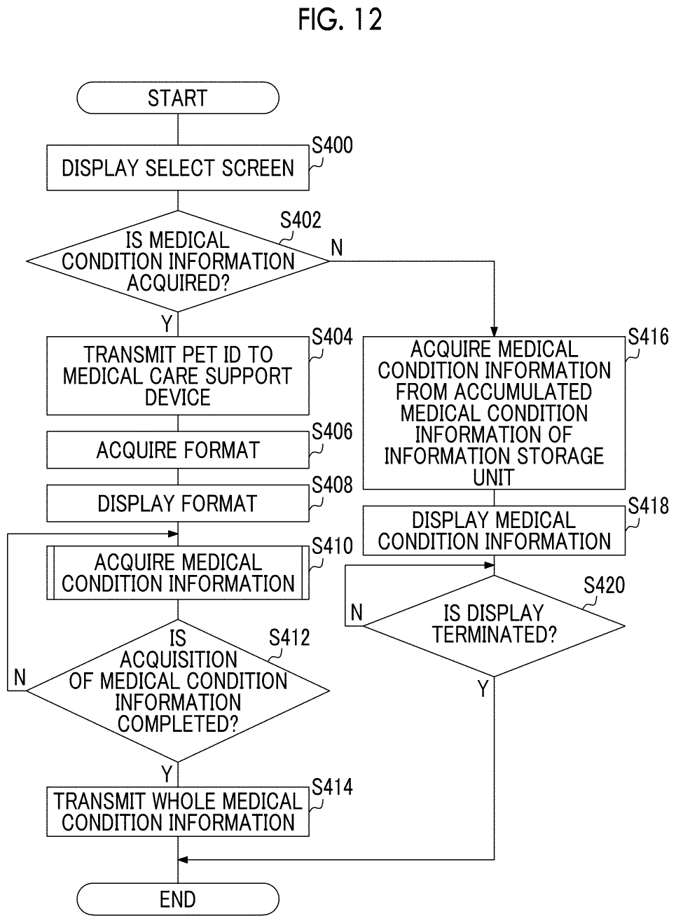

Next, an operation of the owner terminal 14 of this embodiment will be described. In this embodiment, as the CPU 40 of the owner terminal 14 executes the medical condition information acquiring program 43, the medical condition information acquiring process shown in FIG. 12 is executed. As an example, in the medical condition information acquiring process of this embodiment shown in FIG. 12, a process for acquiring medical condition information based on the format 70 acquired from the medical care support device 10 and browsing the accumulated medical condition information is performed.

As an example, in the owner terminal 14 of this embodiment, in a case where the owner U instructs execution of the medical condition information acquiring program 43 registered as an application program using the input unit 46, the medical condition information acquiring process shown in FIG. 12 is executed.

In step S400 of FIG. 12, the acquiring unit 80 causes the display unit 44 to display a selection screen (not shown) for the owner U to select any one of acquisition of the medical condition information 72 or browsing of the medical condition information 72. The owner U selects any one of the acquisition of the medical condition information 72 or the browsing of the medical condition information 72 using the input unit 46 according to the selection screen displayed on the display unit 44.

In the next step S402, the acquiring unit 80 determines whether or not the owner U has selected the acquisition of the medical condition information 72 according to the selection screen displayed in step S400. In a case where the owner U has selected the acquisition of the medical condition information, the determination in step S402 is affirmative, and the procedure proceeds to step S404.

In step S404, the acquiring unit 80 transmits a pet ID for identifying the pet P of the owner U to the medical care support device 10. The pet ID to be transmitted may be registered in advance in the owner terminal 14, or may be input by the owner U through the input unit 46 at the start of the medical condition information acquiring process.

As described above, the output unit 60 of the medical care support device 10 acquires the pet ID transmitted from the owner terminal 14, outputs the format 70 corresponding to the acquired pet ID, and transmits the result to the owner terminal 14 through the network N (steps S118 to S126 in FIG. 11).

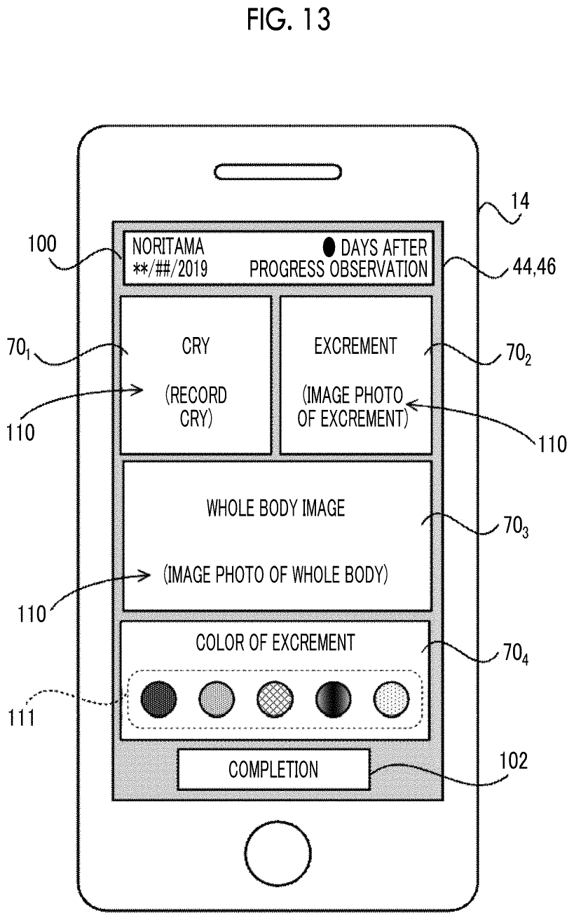

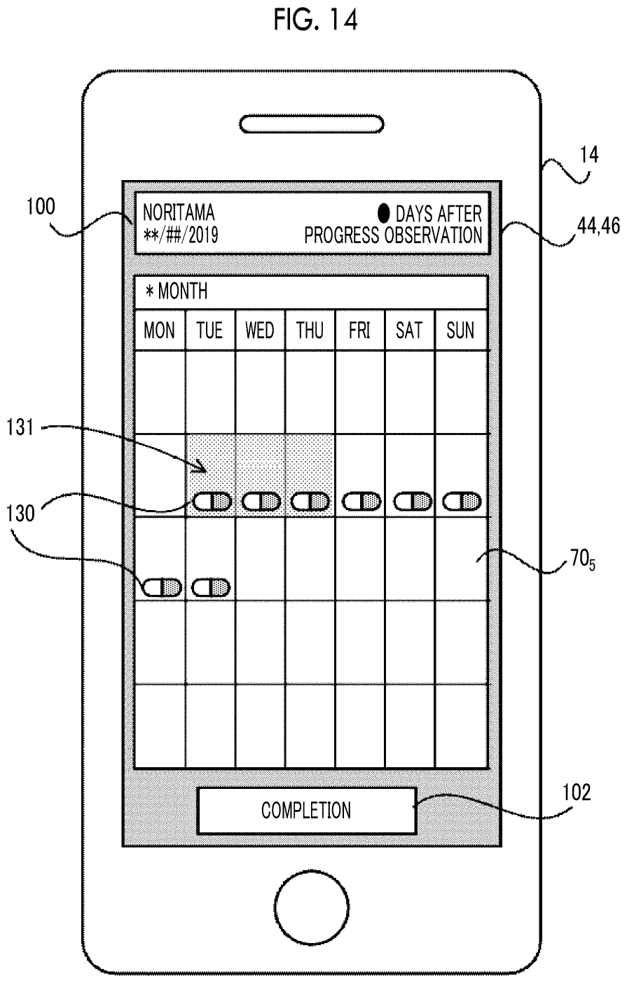

Accordingly, in the next step S406, the acquiring unit 80 acquires the format 70 transmitted from the medical care support device 10. In the next step S408, the acquiring unit 80 causes the display unit 44 to display the acquired format 70. FIGS. 13 and 14 show an example of a state where the format 70 is displayed on the display unit 44 of the owner terminal 14.

The example shown in FIGS. 13 and 14 shows the format 70 (701 to 704) in a case where the pet P has a pet ID “1002” and a name “Noritama” in the identification information/disease name correspondence information 30 shown in FIG. 4. The identification information/disease name correspondence information 30 shown in FIG. 4 indicates that “Noritama” of the pet ID “1002” suffers from “cold”. Since the pet is suffering from “cold”, the format 70 for acquiring “photo (whole body)”, “photo (excrement)”, “cry” and “medication status” is displayed on the display unit 44 of the owner terminal 14 as medical condition information associated with “cold” in the disease name/medical condition information name correspondence information 32 shown in FIG. 5.

The example shown in FIGS. 13 and 14 shows a state where information indicating each of the name of the pet P, current date and time, and the number of days since the progress observation has started is displayed on the display unit 44 of the owner terminal 14 as basic information 100.

Further, the example shown in FIG. 13 shows a state where a format 70₁ for acquiring medical condition information of “cry”, a format 70₂ for acquiring medical condition information of “excrement (photo)”, a format 70₃ for acquiring medical condition information of “photo (whole body)”, and a format 70₄ for acquiring medical condition information of “color (excrement)” are displayed. In addition, as the owner U scrolls the screen of the display unit 44 shown in FIG. 13, as shown in FIG. 14, a format 70₅ for acquiring medical condition information of “medication information” is displayed. In this embodiment, the formats 70₁ to 70₅, and the like are simply referred to as a “format 70” in a case where they are collectively referred to without distinction depending on the medical condition information.

In the next step S410, the acquiring unit 80 performs a medical condition information acquiring process. As the acquiring unit 80 performs the medical condition information acquiring process, the medical condition information is acquired by the owner U according to the format 70.

The acquisition of medical condition information based on the format 70 by the owner U will be described in detail. In the example shown in FIG. 13, each of the formats 70₁ to 70₃ includes a navigation 110 of behaviors to be performed by the owner U in order to acquire the medical condition information. The owner U acts in accordance with the navigation 110 in order to acquire the medical condition information.

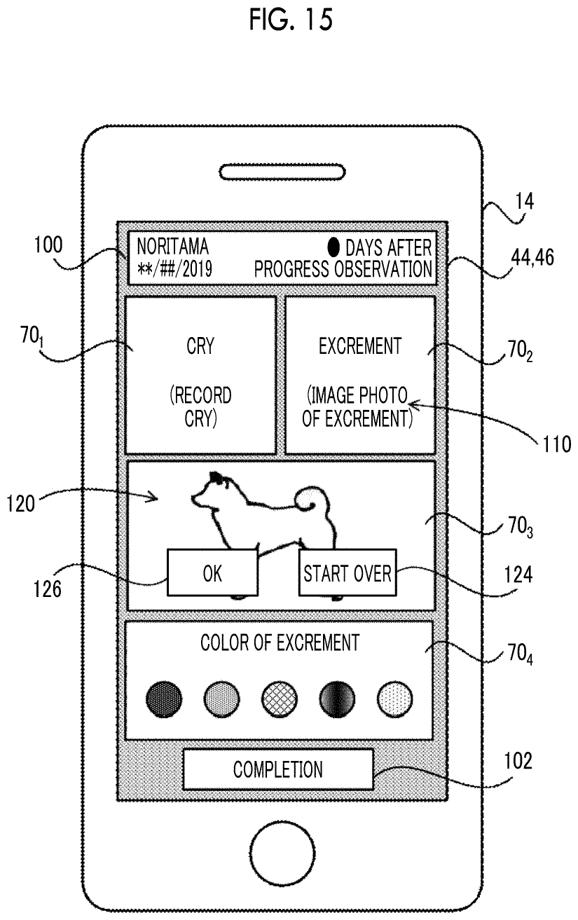

As a specific example, in the owner terminal 14 of the present embodiment, in a case where the medical condition information of “photo (whole body)” is acquired, the owner U instructs (clicks) an area of the format 70₃ displayed on the display unit 44. In a case where it is detected that the area of the format 70₃ has been instructed, the acquiring unit 80 activates the imaging unit 45, and switches the owner terminal 14 to an imaging mode. By switching the owner terminal 14 to the imaging mode, the screen displayed on the display unit 44 is switched to a camera screen on which a subject image is displayed, and the owner U captures a whole body image of the pet P by the imaging unit 45. In a case where the imaging is performed by the imaging unit 45 and a captured image (photo) is acquired, the screen displayed on the display unit 44 by the acquiring unit 80 is switched from the camera screen to a screen on which the format 70 is displayed, of which an example is shown in FIG. 15.

As shown in FIG. 15, in a case where the captured image (photo) is acquired, the acquiring unit 80 displays an acquired photo 120 of the pet Pin the area of the format 70₃ of the display unit 44. Further, the acquiring unit 80 displays buttons 124 and 126 on the photo 120 displayed on the area of the format 70₃. The button 124 is a button operated by the owner U in taking a photo of the pet P again. In a case where it is detected that the button 124 is operated by the owner U, the acquiring unit 80 activates the imaging unit 45, switches the owner terminal 14 to the imaging mode, and repeats the above-described process of acquiring the photo of the pet P.

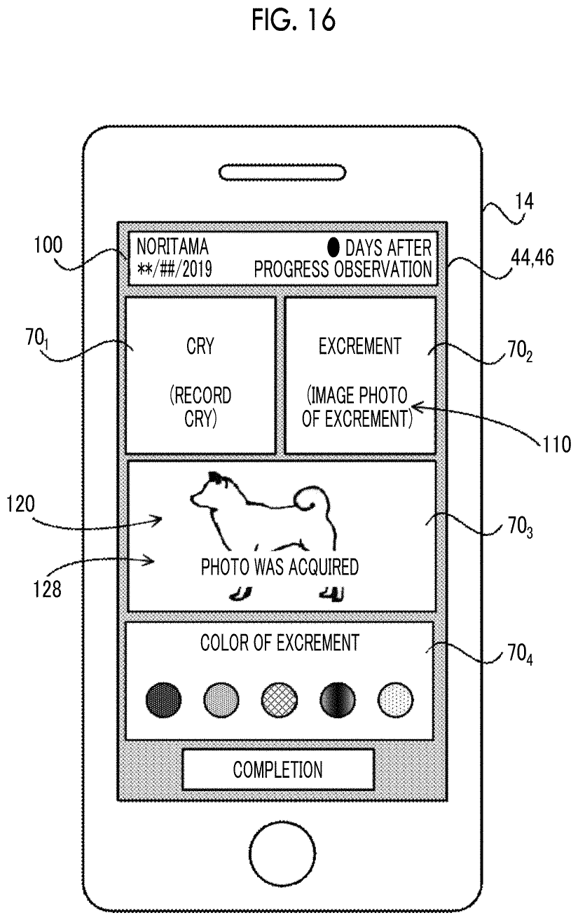

On the other hand, the button 126 is a button operated by the owner U in registering a photo of the pet P as medical condition information. In a case where it is detected that the button 126 is operated by the owner U, the acquiring unit 80 first stores data of the photo 120 in the storage unit 42. Further, as shown in FIG. 16, the acquiring unit 80 replaces the buttons 124 and 126 with completion information 128 indicating that the acquisition of the whole body photo of the pet P is completed on the photo 120 displayed in the area of the format 70₃.

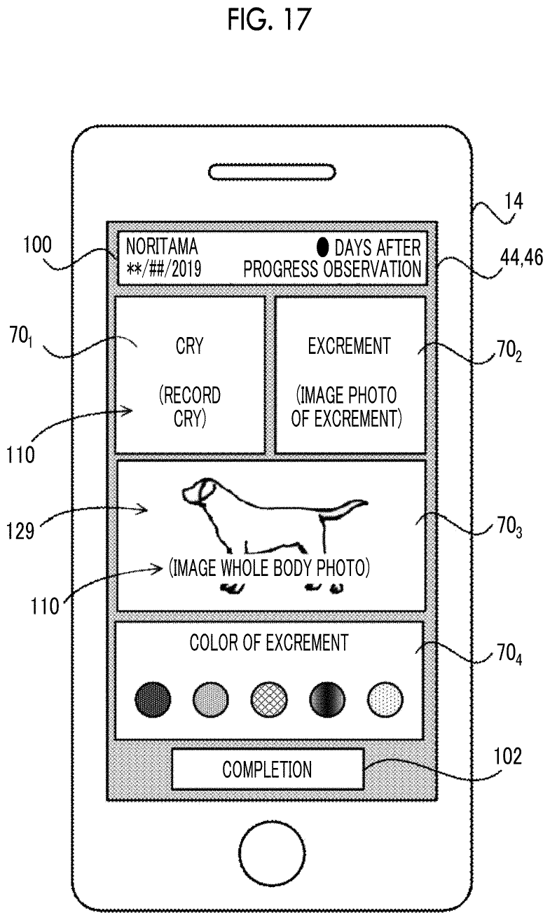

In a case where a captured image (photo) is acquired as the medical condition information in this way, the acquiring unit 80 may display a reference image indicating a state where the medical condition information is acquired as a reference of a method for acquiring the medical condition information in the area of the format 70. In the example shown in FIG. 17, a state where the acquiring unit 80 displays a captured image obtained by imaging a whole body of an animal as a reference image 129 in the format 70₃ is shown. Further, in the above example, a configuration in which the navigation 110 is displayed has been described, but a configuration in which information corresponding to the navigation 110 may be output through the output unit 47 as sound in addition to or in place of the navigation 110 may be used.

Further, for example, in the owner terminal 14 of the present embodiment, in a case where the medical condition information of “cry” is acquired, the owner U instructs (clicks) the area of the format 70₁ displayed on the display unit 44. In a case where it is detected that the area of the format 70₁ has been instructed, the acquiring unit 80 activates a sound collecting device included in the input unit 46, and switches the owner terminal 14 to a sound collecting mode. In a case where the owner terminal 14 is switched to the sound collecting mode, the acquiring unit 80 records sound collected by the sound collecting device for a predetermined time such as one minute, for example. In a case where the sound (cry) is acquired, the acquiring unit 80 displays the buttons 124 and 126 on the format 70₁ in a similar manner to the above-described acquisition of the photo, acquires the sound again in a similar manner to the above description, and performs display of the completion information 128 and storage of the acquired sound data in the storage unit 42.

In addition, in a case where the sound is acquired as the medical condition information in this way, the acquiring unit 80 may activate the imaging unit 45 and may acquire a moving image including sound, instead of acquiring sound using the sound collecting device included in the input unit 46. Further, the acquiring unit 80 may output a sound acquisition method through the output unit 47 as sound.

Further, in the example shown in FIG. 13, a color sample 111 for selecting a color closest to the color of the excrement (stool) of the pet P is included in the format 70₄ for acquiring “color (excrement)” as medical condition information. In the medical care support device 10 of the present embodiment, the owner U selects a color closest to the color of the excrement of the pet P from the plurality of colors shown in the color sample 111, and operates (clicks on) the selected color portion. In a case where it is detected whether or not the operation is performed with respect to any color, the acquiring unit 80 stores the detection result in the storage unit 42.

Further, in the example shown in FIG. 14, the format 70₅ for acquiring “medication information” as medical condition information includes a mark 130 indicating a timing at which the owner U causes the pet P to take a medicine. In the example shown in FIG. 14, the format 70₅ in which the mark 130 is displayed on a medication date on which medication is taken on a calendar. In the example shown in FIG. 14, in a case where the owner U causes the pet P to take a medication, the owner U instructs (clicks) an area corresponding to the date on which the medication is taken. In a case where an instruction from the owner U is detected, the acquiring unit 80 changes an area corresponding to the corresponding date to a color display 131 indicating that the medicine has been taken.

In the example shown in FIG. 14, a configuration in which the mark 130 indicating the timing at which the medicine is taken, that is, the timing at which the medication status is acquired is displayed in a case where the “medication information” is acquired as the medical condition information has been described, but the present invention is not limited to this configuration. For example, in a case where the time corresponds to a timing of medication, such as a date or time of the medication, the acquiring unit 80 may output information indicating that the medication is to be performed, in addition to or in place of the display of the mark 130, through the output unit 47 as sound. Further, in a case where the information indicating that the medication has been performed is not acquired even after the medication timing, in the above example, in a case where a corresponding area is not instructed by the owner U even after the medication date in the format 70₃, it is preferable that the acquiring unit 80 notifies the owner U of information indicating that the medication is not being performed at a predetermined timing.

In a case where the medical condition information corresponding to all the formats 70 is acquired as described above, the owner U instructs (clicks) the completion button 102 shown in FIG. 13, or the like. In this way, in the owner terminal 14 of the present embodiment, the medical condition information is acquired by the owner U on the basis of the format 70.

Accordingly, in a case where it is detected that the completion button 102 has been instructed, the acquiring unit 80 considers that the acquisition of the medical condition information is completed. In step S412 of FIG. 12, the acquiring unit 80 determines whether or not the acquisition of the medical condition information is completed. Until the acquisition of the medical condition information is completed, the determination in step S412 is negative. On the other hand, in a case where the acquisition of the medical condition information is completed, the determination in step S412 is affirmative, and the procedure proceeds to step S414.

In step S414, the acquiring unit 80 transmits all the medical condition information 72 acquired by the process of step S410 to the medical care support device 10 through the network N, and then, terminates the medical condition information acquiring process.

On the other hand, in a case where the owner U selects the browsing of the medical condition information 72 on the selection screen displayed on the display unit 44 in step S400, the process in step S402 is negative, and the procedure proceeds to step S416.

In step S416, the browsing unit 82 acquires medical condition information corresponding to the pet ID from the accumulated medical condition information 36 in the information storage unit 12 through the network N.

In the next step S418, the browsing unit 82 causes the display unit 44 to display the acquired medical condition information. Thus, the owner U can browse a history of the medical condition information of the pet P, that is, a progress state of the medical condition information. In the owner terminal 14 of this embodiment, in a case where the display of the medical condition information is to be terminated, the owner U gives an instruction for terminating the display through the input unit 46.

In the next step S420, a negative determination is made until the browsing unit 82 detects that the owner U has instructed to terminate the display. On the other hand, in a case where the browsing unit 82 detects that the owner U has instructed to terminate the display, the determination in step S420 is affirmative, and the medical condition information acquiring process is terminated.

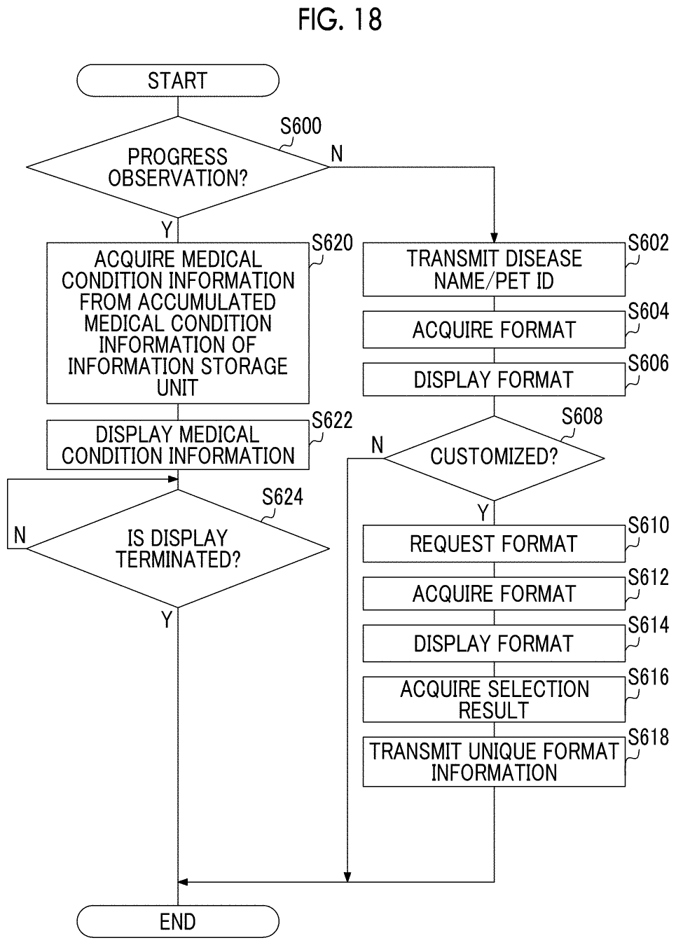

Next, an operation of the veterinarian terminal 16 of this embodiment will be described. In this embodiment, as the CPU 50 of the veterinarian terminal 16 executes the progress observation program 53A, a progress observing process shown in FIG. 18 is executed. As an example, in the progress observing process of this embodiment shown in FIG. 18, a process for customization by the veterinarian V for a format used for acquiring medical condition information and observation of accumulated medical condition information is performed.

As an example, in the veterinarian terminal 16 of the present embodiment, in a case where the veterinarian V instructs the input unit 56 to perform a process related to a progress observation, the progress observing process shown in FIG. 18 is executed.

In step S600 of FIG. 18, the customization unit 90 determines whether or not to perform a progress observation based on the accumulated medical condition information. In a case where the veterinarian V inputs an instruction for customizing the format through the input unit 56, the determination in step S600 is negative, and the procedure proceeds to step S602. In step S602, the customization unit 90 transmits a disease name or a pet ID that is a target of format customization, which is input by the veterinarian V using the input unit 56.

As described above, in a case where the medical care support device 10 receives the disease name or the pet ID transmitted from the veterinarian terminal 16, the output unit 60 transmits a format according to the received disease name or pet ID to the veterinarian terminal 16 (see FIG. 11, steps S104 to S108).

Accordingly, in the next step S604, the customization unit 90 acquires the format received by the veterinarian terminal 16 from the medical care support device 10 through the network N. In the next step S606, the customization unit 90 displays the acquired format on the display unit 54. The veterinarian V performs customization in a desired format with reference to the format displayed on the display unit 54. In a case where the veterinarian V wants to customize the format, the veterinarian V instructs the customization of the format through the input unit 56.

In step S608, the customization unit 90 determines whether or not a customization instruction has been given. In a case where the customization instruction has not been given within a predetermined time, the determination in step S608 is negative, and the progress observing process is terminated. On the other hand, in a case where the customization instruction has been given, the process in step S608 is affirmative, and the procedure proceeds to step S610.

Examples of the customization include changing the format associated with the disease name. A specific example of this case will be described with reference to FIG. 5. For example, in the example shown in FIG. 5, “photo (whole body)”, “photo (affected portion)”, and “medication information” are associated with “dermatitis”, as the medical condition information name. Accordingly, in the example shown in FIG. 5, for the pet P that is suffering from dermatitis, formats corresponding to “photo (whole body)”, “photo (affected portion)”, and “medication information” are displayed in step S606. In a case where the veterinarian V also uses “cry” as medical condition information of the pet P that is suffering from dermatitis for medical care, the veterinarian V instructs the input unit 56 to make a request for a format corresponding to “cry”.

In addition, for example, regarding a disease from which the pet P is suffering, the previous medical examination has diagnosed that the pet is suffering from “dermatitis”, but it is suspected that the pet is suffering from “parasite”, the veterinarian V may register the format of the medical condition information corresponding to both diseases as unique format information of the pet P. In the example shown in FIG. 5, as the unique format information of the pet ID transmitted in step S602, information indicating that the formats of “photo (excrement)”, “odor (excrement)”, and “color (excrement)” associated with “parasite” are added, in addition to the formats of “photo (whole body)”, “photo (affected portion)”, and “medication information” associated with “dermatitis”, may be registered. In this case, since the format corresponding to “dermatitis” is displayed on the display unit 54 in step S606, the veterinarian V instructs the input unit 56 to request the format corresponding to “parasite”.

In step S610, the customization unit 90 requests a format according to the instruction from the veterinarian V. Specifically, the customization unit 90 transmits a request for a format corresponding to the medical condition information desired by the veterinarian V to the medical care support device 10 through the network N.

As described above, in a case where the medical care support device 10 receives the request transmitted from the veterinarian terminal 16, the output unit 60 transmits the format according to the received request to the veterinarian terminal 16 (see FIG. 11, steps S110 and S112).

Accordingly, in the next step S612, the customization unit 90 acquires the format received by the veterinarian terminal 16 from the medical care support device 10 through the network N. In the next step S614, the customization unit 90 further displays the format acquired in step 5612 on the display unit 54 on which the format has already been displayed by the process of step S606.

The veterinarian V selects a format to be registered in association with the disease name or pet ID from the format displayed on the display unit 44 using the input unit 56.

In the next step S616, the customization unit 90 acquires the selection result of the veterinarian V. In the next step S618, the customization unit 90 transmits the selection result of the veterinarian V as unique format information, and then, the progress observing process is terminated.

As described above, in a case where the medical care support device 10 receives the unique format information transmitted from the veterinarian terminal 16, the output unit 60 registers the received unique format information (see FIG. 11, steps S114 and S116).

In this embodiment, a configuration in which in a case where the format is customized, a format associated with medical condition information is changed has been described, but the invention is not limited to this configuration. For example, a configuration in which a display format such as a layout, or display content is customized (changed) with respect to a certain format may be used.

On the other hand, in a case where the veterinarian V inputs an instruction for performing a progress observation based on accumulated medical condition information through the input unit 56, the determination in the process of step S600 is affirmative, and the procedure proceeds to step S620.

In step S620, the progress observation unit 94 acquires the medical condition information corresponding to the pet ID from the accumulated medical condition information 36 in the information storage unit 12 through the network N.

In the next step S622, the progress observation unit 94 displays the acquired medical condition information on the display unit 54. Thus, the veterinarian V can observe a history of the medical condition information of the pet P, that is, can perform the progress observation of the medical condition information. As an example, in the veterinarian terminal 16 of the present embodiment, in a case where the display of the medical condition information is to be terminated, the veterinarian V gives an instruction for terminating the display through the input unit 56.

In the next step S624, a negative determination is made until the progress observation unit 94 detects that the veterinarian V has instructed the display to be terminated. On the other hand, in a case where the progress observation unit 94 detects that the veterinarian V has instructed to terminate the display, the determination in step S624 is affirmative, and the progress observing process is terminated.

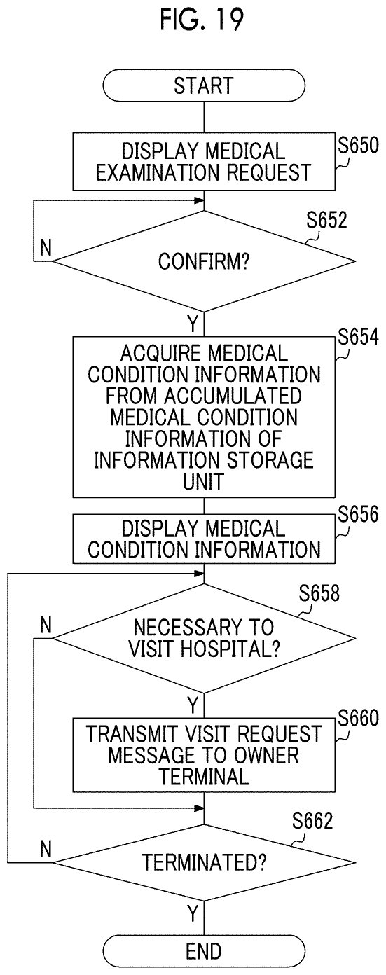

Further, in this embodiment, as the CPU 50 of the veterinarian terminal 16 executes the medical examination request program 53B, the medical examination requesting process shown in FIG. 19 is executed. As an example, in the medical examination requesting process of this embodiment shown in FIG. 19, as described above, in a case where the veterinarian terminal 16 receives a medical examination request (see FIG. 11, step S134) transmitted from the medical care support device 10 through the network N, the medical examination requesting process shown in FIG. 19 is executed.

In step S650 of FIG. 19, the medical examination instruction unit 92 displays the received medical examination request on the display unit 54. A method for displaying the medical examination request by the medical examination instruction unit 92 is not particularly limited. For example, the medical examination instruction unit 92 may display a message indicating the medical examination request on the display unit 44 that is a liquid crystal display of the veterinarian terminal 16, or may display a message indicating the medical examination request as sound. As an example, in a case where the veterinarian V wants to confirm the medical examination request from the medical examination instruction unit 92, the veterinarian V gives an instruction for confirming the medical examination request through the input unit 56.

The medical examination instruction unit 92 makes a negative determination in step S652 until the instruction for confirming the medical examination request from the veterinarian V is input through the input unit 56. On the other hand, in a case where the instruction for confirming the medical examination request from the veterinarian V is input through the input unit 56, the determination in step S652 is affirmative, and the procedure proceeds to step S654. In step S654, the medical examination instruction unit 92 acquires the medical condition information corresponding to the pet ID for which medical examination is requested from the accumulated medical condition information 36 in the information storage unit 12 through the network N.

In the next step S656, the medical examination instruction unit 92 displays the acquired medical condition information on the display unit 54. The veterinarian V confirms a history of the medical condition information of the pet P displayed on the display unit 54, that is, the medical condition information. Further, the veterinarian V confirms whether or not a sudden change or the like occurs in the medical condition of the pet P, and confirms whether or not it is necessary to visit the hospital (or to see the doctor) without waiting for an appointment date for the next visit. Further, in a case where the veterinarian V determines that the visit is necessary, the veterinarian V gives an instruction indicating that the visit is necessary through the input unit 56.

In the next step S658, the medical examination instruction unit 92 determines whether or not the instruction indicating that the visit is necessary is input by the veterinarian V through the input unit 56. In a case where the instruction indicating that the visit is necessary is not input by the veterinarian V through the input unit 56, the determination in step S658 is negative, and the procedure proceeds to step S662. On the other hand, in a case where the instruction indicating that the visit is necessary is necessary is not input by the veterinarian V through the input unit 56, the determination in step S658 is affirmative, and the procedure proceeds to step S660.

In step S660, the medical examination instruction unit 92 transmits a message for requesting a predetermined visit to the owner terminal 14 of the owner U corresponding to the pet P.

In the next step S662, the medical examination instruction unit 92 determines whether or not to terminate the medical examination requesting process. As an example, in the veterinarian terminal 16 according to the present embodiment, the veterinarian V gives an instruction for terminating the confirmation through the input unit 56 in a case where the confirmation of the medical condition information is terminated. Until the medical examination instruction unit 92 detects that the veterinarian V gives the instruction for terminating the confirmation, the determination in step S662 is negative, and the procedure returns to step S658, and then, the processes of steps S658 and S660 are repeated. On the other hand, in a case where the medical examination instruction unit 92 detects that the veterinarian V has given the instruction for terminating the confirmation, the determination in step S662 is affirmative, and the medical examination requesting process is terminated.

As described above, the medical care support device 10 according to the present embodiment includes the output unit 60 that outputs a format which is support information for supporting acquisition, by the owner U of the pet P, of medical condition information indicating a medical condition of the disease from which the pet P suffers, and the acquiring unit 62 that acquires the medical condition information acquired by the owner U on the basis of the format.

Thus, in the medical care support device 10 of the present embodiment, it is possible to acquire the medical condition information acquired by the owner U according to the format output from the medical care support device 10. Particularly, even in a case where it is difficult for the pet P itself to send the medical condition information as information necessary for medical care of the disease to the owner U or the veterinarian V, it is possible to appropriately acquire the medical condition information according to the disease.

Accordingly, according to the medical care support device 10 of the present embodiment, the veterinarian V can use the acquired medical condition information for medical care. For example, the veterinarian V can acquire the medical condition information for a period until the next visit after the pet P visits the animal hospital. In order to obtain the medical condition information continuously during the period until the hospital visit, for example, the veterinarian V can determine whether there is simply no change even in a case where there is no change in the medical condition of the pet P between the previous visit and the current visit, or the medical condition is recovered after the medical condition gets worse after the visit. Accordingly, according to the medical care support device 10 of the present embodiment, it is possible to improve efficiency of medical care.

In this embodiment, a configuration in which the medical care support system 1 corresponds to one owner terminal 14 has been described, but a configuration in which the medical care support system 1 manages a plurality of owner terminals 14 may be used. Further, for example, a configuration in which the medical care support system 1 corresponds to a plurality of veterinarian terminals 16 and further veterinarian terminals 16 of a plurality of animal hospitals and supports diagnosis of a pet P that is a patient in a plurality of hospitals may be used. As the information storage unit 12 accumulates the medical condition information of the pet P in the plurality of animal hospitals as the accumulated medical condition information 36, for example, even in a case where a home hospital of the pet P is replaced, it is possible to use the accumulated medical condition information, and thus, it is possible to further improve efficiency of medical care by the veterinarian V.

Further, in this embodiment, a configuration in which a part of the functions of the medical care support device 10, for example, at least a part of the functions of the output unit 60, the acquiring unit 62, and the request unit 64 are provided in another device such as the owner terminal 14, instead of or together with the medical care support device 10 may be used. Further, for example, the information storage unit 12 may be provided in the medical care support device 10. Furthermore, for example, a configuration in which a part or all of the identification information/disease name correspondence information 30, the disease name/medical condition information name correspondence information 32, the format information 34, and the accumulated disease information 36 stored in the information storage unit 12 are stored in another device may be used.

Further, in this embodiment, a configuration in which the medical care support system 1 supports the medical care of the pet P has been described, but the invention may be applied to a configuration in which the medical care support system 1 (medical care support device 10) supports, for example, human medical care other than the pet P. Even in a case where the pet P is a human being, it is possible to improve efficiency of medical care in the same manner as described above, and in particular, to improve efficiency of medical care for infants who are not good at expressing their medical conditions (symptoms).

Further, in the above embodiment, various processes executed as the CPU executes software (programs) may be executed by various processors other than the CPU. In this case, as the processors, a programmable logic device (PLD) of which a circuit configuration is changeable after manufacturing, such as a field-programmable gate array (FPGA), a dedicated electric circuit that is a processor having a circuit configuration that is exclusively designed to execute a specific process, such as an application specific integrated circuit (ASIC), and the like are exemplified. Further, the various processes may be executed by one of the various processors, or may be executed by combination of two or more processors of the same type or different types (for example, combination of a plurality of FPGAs or combination of a CPU and an FPGA). In addition, a hardware structure of the various processors is, more specifically, an electric circuit in which circuit elements such as semiconductor elements are combined.

Further, in the above-described embodiment, a configuration in which the support program 23 is stored (installed) in the storage unit 22 in advance, the medical condition information acquiring program 43 is stored in the storage unit 42 in advance, and the progress observation program 53A and the medical examination request program 53B are stored in the storage unit 52 in advance has been described, but the invention is not limited thereto. Each of the support program 23, the medical condition information acquiring program 43, the progress observation program 53A, and the medical examination request program 53B may be provided in a state of being recorded in a recording medium such as a compact disc read only memory (CD-ROM), a digital versatile disc read only memory (DVD-ROM), or a universal serial bus (USB) memory. Further, each of the support program 23, the medical condition information acquiring program 43, the progress observation program 53A, and the medical examination request program 53B may be downloaded from an external device through a network.

According to a second aspect of the present disclosure, in the medical care support device according to the first aspect of the present disclosure, a correspondence relationship between a type of the disease and the medical condition information to be acquired is determined in advance, and the processor outputs the support information for supporting the acquisition of the medical condition information depending on the type of the disease, on the basis of the correspondence relationship.

According to a third aspect of the present disclosure, in the medical care support device according to the second aspect of the present disclosure, the processor outputs medical condition information selected from medical condition information that is not associated with the type of the disease in the correspondence.

According to a fourth aspect of the present disclosure, in the medical care support device according to any one aspect of the first to third aspects of the present disclosure, the medical condition information includes at least one of information related to an appearance of the pet, information related to a behavior of the pet, or information related to excrement.

According to a fifth aspect of the present disclosure, in the medical care support device according to any one aspect of the first to third aspects of the present disclosure, the medical condition information includes at least one of an image of a whole body, an image of an affected portion, an image of excrement, a cry, a color of excrement, an odor of excrement, or medication information.

According to a sixth aspect of the present disclosure, in the medical care support device according to any one aspect of the first to fifth aspects of the present disclosure, the support information includes at least one of a method for acquiring the medical condition information or a timing at which the medical condition information is acquired.

According to a seventh aspect of the present disclosure, in the medical care support device according to any one aspect of the first to sixth aspects of the present disclosure the processor requests a veterinarian for medical care at a predetermined timing based on at least one of the disease or the medical condition information.

According to an eleventh aspect of the present disclosure, an information processing apparatus according to the present disclosure comprises includes a memory that stores a command for execution of a computer and a processor configured to execute the stored command, in which the processor outputs support information for supporting acquisition, by an owner of a pet, of medical condition information indicating a medical condition of a disease from which the pet suffers and acquires information of the pet acquired by the owner on the basis of the support information.

According to the present disclosure, it is possible to improve efficiency of medical care.

What is claimed is:

1. A medical care support device comprising a processor that is configured to:
output support information for supporting acquisition, by an owner of a pet, of medical condition information indicating a medical condition of a disease from which the pet suffers; and
acquire information of the pet acquired by the owner on the basis of the support information.

2. The medical care support device according to claim 1,
wherein a correspondence relationship between a type of the disease and the medical condition information to be acquired is determined in advance, and
wherein the processor outputs the support information for supporting the acquisition of the medical condition information depending on the type of the disease, on the basis of the correspondence relationship.

3. The medical care support device according to claim 2,
wherein the processor outputs medical condition information selected from medical condition information that is not associated with the type of the disease in the correspondence.

4. The medical care support device according to claim 1,
wherein the medical condition information includes at least one of information related to an appearance of the pet, information related to a behavior of the pet, or information related to excrement.

5. The medical care support device according to claim 2,
wherein the medical condition information includes at least one of information related to an appearance of the pet, information related to a behavior of the pet, or information related to excrement.

6. The medical care support device according to claim 3,
wherein the medical condition information includes at least one of information related to an appearance of the pet, information related to a behavior of the pet, or information related to excrement.

7. The medical care support device according to claim 1,
wherein the medical condition information includes at least one of an image of a whole body, an image of an affected portion, an image of excrement, a cry, a color of excrement, an odor of excrement, or medication information.

8. The medical care support device according to claim 2,
wherein the medical condition information includes at least one of an image of a whole body, an image of an affected portion, an image of excrement, a cry, a color of excrement, an odor of excrement, or medication information.

9. The medical care support device according to claim 3,
wherein the medical condition information includes at least one of an image of a whole body, an image of an affected portion, an image of excrement, a cry, a color of excrement, an odor of excrement, or medication information.

10. The medical care support device according to claim 1,
wherein the support information includes at least one of a method for acquiring the medical condition information or a timing at which the medical condition information is acquired.

11. The medical care support device according to claim 2,
wherein the support information includes at least one of a method for acquiring the medical condition information or a timing at which the medical condition information is acquired.

12. The medical care support device according to claim 3,
wherein the support information includes at least one of a method for acquiring the medical condition information or a timing at which the medical condition information is acquired.

13. The medical care support device according to claim 1, further comprising:
a request unit that requests a veterinarian for medical care at a predetermined timing based on at least one of the disease or the medical condition information.

14. The medical care support device according to claim 2, further comprising:
a request unit that requests a veterinarian for medical care at a predetermined timing based on at least one of the disease or the medical condition information.

15. The medical care support device according to claim 3, further comprising:
a request unit that requests a veterinarian for medical care at a predetermined timing based on at least one of the disease or the medical condition information.

16. A medical care support method comprising:
by one or more processors:
outputting support information for supporting acquisition, by an owner of a pet, of medical condition information indicating a medical condition of a disease from which the pet suffers; and
acquiring information of the pet acquired by the owner on the basis of the support information.

17. A non-transitory computer-readable storage medium storing a medical care support program for causing a computer to:
output support information for supporting acquisition, by an owner of a pet, of medical condition information indicating a medical condition of a disease from which the pet suffers; and
acquire information of the pet acquired by the owner on the basis of the support information.

18. A terminal device comprising:
a display device that displays support information for supporting acquisition, by a user, of medical condition information indicating a medical condition of a disease from which the pet suffers; and
an input device that accepts input information from the user on the basis of the support information.
